(19) United States
(12) Patent Application Publication
(10) Pub. No.: US 2018/0110900 A1
(43) Pub. Date: Apr. 26, 2018

(54) Therapeutic Applications of Honey and Amniotic Membrane for the Treatment of Disease

(71) Applicant: Michael S. Korenfeld, Wildwood, MO (US)

(72) Inventor: Michael S. Korenfeld, Wildwood, MO (US)

(21) Appl. No.: 15/788,771

(22) Filed: Oct. 19, 2017

Related U.S. Application Data
(60) Provisional application No. 62/377,630, filed on Aug. 21, 2016.

(51) Int. Cl. A61L 27/36; A61K 9/00; A61K 35/50; A61K 35/644; G02B 1/04; A61L 27/54

(52) U.S. Cl. CPC A61L 27/3604; A61K 9/0051; A61K 35/50; A61L 27/54; G02B 1/043; A61K 9/0048; A61L 27/3691; A61K 35/644

(57) ABSTRACT

A therapeutic material or pharmaceutical is composed of 1) amniotic membranes in a large intact sheet, 2) lysates of intact sheets of amniotic membrane, or 3) the non-particulate fluids produced while creating lysates of intact sheets of amniotic membrane. Any of the embodiments of the therapeutic material can be formed with or without the incorporation of complex chemicals like hyaluronic acid and/or honey and/or additional therapeutic cells. The device or fluid is used to treat diseased tissue. Lysates of the amniotic membrane can be formed into any desirable shape, and then dehydrated, to maintain the desired 3D form of the device.

### CROSS-REFERENCE TO RELATED APPLICATION

This application relies upon, and claims the benefit of the filing date of, U.S. Provisional Patent Application Ser. No. 62/377,630, filed Aug. 21, 2016, the entirety of which is incorporated herein by reference. A petition under 37 CFR §1.78(b) accompanies this application, along with the fee therefor

### BACKGROUND OF THE INVENTION

### Field of the invention

The present invention relates, generally, to the use of amniotic tissues to treat diseased tissues either externally or internally, and, more particularly, to the use of lysates of amniotic membranes, in fluid or solid shapes, and intact sheets of amniotic membranes, with or without the addition of honey to achieve a therapeutically beneficial clinical synergy of the intrinsic medicinal properties of both primary contributing elements.

### Background of the Invention

The amniotic membrane (AM) not only has a structural function in the womb, it also is rich in growth factors and other healthful cells and chemicals that ensure the developing fetus has a nutrient rich environment in which to develop. Additionally, because the amniotic fluid is an ultra-filtrate of the blood, it contains many of the constituents found in plasma and serum in concentrations that are ideal for healthy biological function of tissues.

For purposes of providing background information relative to the present invention, and as stated again hereafter, it is to be understood that the term “AM” is used herein, with reference to the present invention, as an abbreviation for amniotic membrane, and is further intended to include amniotic tissue of any kind, chorionic membrane, amniotic fluid, the umbilical cord and the placenta. This use and definition of AM is understood to apply throughout this application, including the detailed description and claims.

Many companies presently acquire the amniotic membrane (“AM”) from baby deliveries, ensure that the mother and offspring do not have contagious infectious diseases, and then process the AM for therapeutic purposes. For example, AM can be processed as an intact sheet, and offered as a planar, dried, and sterile product for use in reconstruction of the ocular surface after such operations as pterygium (fibrovascular growth) removal. In known ophthalmic surgery the sheet size of AM is determined by the surgeon, and then either sewn to the ocular surface or glued into place with fibrin glue. This AM transplant hastens the healing process, and has been shown to greatly reduce the pterygium recurrence rate, when compared to the same surgery performed without an AM transplant.

Other companies have harvested thin, intact sheets of AM and processed them so that they are attached in a planar fashion to a rigid ring of plastic that is placed onto a damaged eye. The AM remains in contact with the ocular surface over 3-5 days. This helps the eye recover from various insults that are traumatic, infectious, or autoimmune in nature. Once the AM has been dissolved out of the carrier ring, the ring is removed.

Still other manufacturers harvest intact AM, and make intact sterile lyophilized sheets of it available for placement under a conventional soft contact lens, which holds it in place while it is working. In this method, the AM is too thin, even when hydrated, to remain in residence on the eye and quickly becomes dislocated from its intended location without the use of the conventional contact lens. With this known strategy, the AM film will always fold when placed onto the curved eyeball, because of the mismatch in shapes, even when a conventional contact lens is placed over it.

All current commercially available amniotic membrane preparations that are lyophilized are formed of a single intact sheet of amniotic membrane. This is desirable for many applications, but a single intact sheet is film-like, very thin and hard to handle. It can even be accidentally moved by ambient air currents and static electricity into undesirable positions during handling. Also, a single small sheet of amniotic membrane is substantially planar. Because it has very little volume, it cannot be shaped into multiple forms during manufacturing or in the field during use, and it is relegated to covering some exterior body surface.

AM is well-known for use on the eyeball when there is some type of disease or adverse condition. The placement of a conventional contact lens on top of the AM is not ideal, since the surface of the eyeball gets most of its oxygen from atmospheric gas. The contact lens therefore imparts a lower oxygen tension to the diseased tissues. This impairs wound healing and the immune response to the diseased tissues. The tissues of the surface of the eyeball also get much of their nutrition from the tears of the eye. The tears are also responsible for the removal of cellular waste from the ocular surface. A conventional contact lens disrupts the tear film, and reduces the performance of these important biological activities. Additionally, a conventional contact lens creates a surface for a biofilm to develop over time, and this will increase the risk of tissue infection, especially when the tissue that is being treated is already devitalized. The known contact lenses that are used to hold AM in place are not removed for several days, so there is ample time for a biofilm to develop. Despite these shortcomings that are inherent in the use of a conventional contact lens over an AM placed upon the globe of the eye, this method of AM application is successful at speeding the healing and recovery of a wide range of ocular surface disorders.

Currently, an FDA-approved product known as a “collagen shield” has been made and sold in the US. The collagen shield is provided as a dehydrated “contact lens shaped” thin sheet in a sterile package. In use, the collagen shield is hydrated with a solution, most typically a topical antibiotic, that not only renders the collagen shield soft and malleable, but also absorbs and adsorbs the antibiotic, and holds it in direct approximation to the tissues being treated, ensuring a high and continuous dose of antibiotic, while simultaneously acting as a collagen template for wound healing promotion. Current bandage contact lenses are made of porcine collagen and are designed to dissolve in 8-24 hours, a much shorter time period than is found in current AM film preparations.

Another known tissue-based product presently used for medical applications is autologous serum eye drops (ASEDs). Unlike the AM preparations, which are harvested from donors that are unrelated to the recipients, ASEDs are prepared from the blood of the same person who ultimately receives the prepared eye drops. The blood is drawn, clotted, and spun, and then the serum is removed and placed directly into sterile bottles for distribution back to the patient, or first diluted by sterile physiologic solution to a certain prescribed concentration, and then aliquoted into sterile eye drop bottles for distribution back to the donor-patient. The fact that this tissue is autologous makes its compatibility with the end user guaranteed and safe.

With AM, the tissue does not express Class II antigens, and therefore is very unlikely to induce an immune reaction. Despite careful screening of AM donors, it still remains at least possible that the donor will transmit some infectious disease to the end user, where this is impossible with ASEDs. Similar strategies are being employed with blood that has not been clotted, and is referred to as Platelet-Rich Plasma. It offers the same kinds of advantages as ASEDS: a complex therapeutic fluid that contains a broad range of electrolytes, growth factors, vitamins, hormones, and nutrients that represent the vital constituents of blood. These biologically derived therapeutic fluids are vastly richer in the spectrum of constituents that are needed for cellular health as compared to any formulation that could be conceived by a manufacturer, formulated by adding a limited number of specific chemicals to water, tested in a clinical trial, and then become FDA-approved.

Although matching the donor and recipient of ASEDs ensures the safety of the product, the primary problem with ASEDs is the high transactional cost associated with the harvesting and processing of small batches. AM, on the other hand, can be harvested in larger quantities, and processed in bulk, leading to reduced transactional costs for their preparation. Considering the demonstrated efficacy of AM for a host of applications and the safety of the product in wide spread use, additional products derived from AM are desirable, even if they become characterized and regulated differently by the FDA as drugs or devices, and not just “minimally manipulated” biologics.

Honey is a substance long deemed to have healthful properties and to be generally very stable and not disposed to easy spoilage in its natural condition. A patent application by Maloney, published Sep. 16, 2010 (US2010/0233283 A1) describes the use of a uni-floral, non-peroxide activity (NPA) honey, specifically derived from plants in the genus Leptospermum that can be used as an eye drop, and formulated to contain a dilution of approximately 19-80% water.

Honey has been known to help heal wounds, and by virtue of its chemical nature, has also been known to have antibacterial properties. Some of these properties derive from its peroxide activity (PA), while other antibacterial properties derive from non-peroxide activity (NPA). Honeys that have strong non-peroxide activity are largely derived from nectars and pollens collected from plants within the Genus Leptospermum, in the myrtle Family, Myrtaceae.

Manuka honey and jelly bush honey are an example of these NPA honeys. Honey that is present within the capped cells of a bee hive are kept at a specific water percentage, which confers many of the honey's useful properties, but also keeps the honey from fermenting through the growth of yeast, which ubiquitously has resident spores in the honey, but which are unable to grow, because of the approximately 18% water content of this hygroscopic fluid. When most honeys take on more than approximately 18% water, the yeast spores are enabled to grow, and the yeast ferments the honey, and “spoils” it. An exception to this characteristic of honey is found in the uni-floral honeys (honeys derived from a single specific plant source) derived from plants within the Genus Leptospermum, in the myrtle Family, Myrtaceae. These honeys retain their ability to resist fermentation by yeast, even when they are diluted by water up to at least 25%, and maintain a shelf life of at least a year. Additional dilution is also tolerated beyond the 25% level.

Honey with peroxide activity (PA honey) also has many desirable attributes, including antibacterial activity, but PA honey suffers the risk of spoilage if its water content rises above 18%. Thus use of PA honey requires addition of a preservative to prevent the germination of fungi, or the growth of other microorganisms, in order to allow it to be diluted to a solution that is 19-99% water. Once such an aqueous honey solution is created, it can be used as an eye drop to treat dry eyes and infectious conditions that affect the conjunctiva and/or cornea Likewise, a NPA honey that is diluted to a level above 80% water can also be preserved by chemical additives if necessary.

In its native state, honey can be further dehydrated to remove water, so that the aqueous concentration can be lowered from 18% to 0%, forming an anhydrous honey powder. Honey that exists in concentrations below 18% is also of medicinal value, and is included in the useful elements of the present invention. Honey with an aqueous concentration lower than 18% would be naturally antimicrobial, even to a greater extent than 18% water content honey. Nevertheless, preservatives that prevent bacterial and fungal growth can also be added to honey with less than 18% water, as added assurance of the prevention of decay. The present invention includes a new usage for honeys, as described further below.

### SUMMARY OF THE INVENTION

To date, there are no solid AM preparations that have a thickness that is greater than the intrinsic thickness of a single sheet of AM, which is typically thinner than 100 microns. This limits the range of applications for this amazing tissue. Thus, there is a need in the medical marketplace for a thicker and/or more alterable and more functional form of AM.

One of the primary applications for the new AM lysates molded, fenestrated or un-fenestrated, and formed as a dehydrated contact lens, is for use after cataract surgery. This AM device will promote rapid healing, deliver a high concentration of selected antibiotic and/or anti-inflammatory drugs directly to the globe of the eye, act as a mechanical barrier to prevent the entrance or egress of materials into and out of the incised globe, and offer intrinsic anti-biosis when the product incorporates honey. Many other applications for this kind of contact lens can be contemplated for any ocular disorder that requires antibiosis, enhanced healing efficacy, and anti-inflammatory activity.

Molded 3D products formed using the methods described below can be deployed on the skin, on the scalp, on the anus, on the vaginal introitus, or on the lips. Molded 3D products formed using these methods can also be deployed internally, in the mouth, along the gastrointestinal tract, along the lumens of the female reproductive tract, in joint spaces, and in surgery where tissue planes are cut in any internal organ system in the body.

Accordingly, the present invention includes, briefly, a three-dimensional (3D) device for treatment of diseased tissues by application of the 3D device to such diseased tissues. The device is made of micronized amniotic membrane (AM) tissue in a physiologic solution and dehydrated to a paste-like consistency for selective manipulation into a specific preselected form.

The invention is also, briefly a therapeutic fluid for treatment of diseased tissues by application of the therapeutic fluid to such diseased tissues. The therapeutic fluid is formed from liquids generated and separated during micronization of amniotic membranes (AM).

The invention is further, briefly, a method of making a therapeutic material for treatment of diseased tissues. The method includes the steps of: providing amniotic membrane (AM) tissue, providing a physiologic solution, mixing the AM tissue and the physiologic solution, and micronizing the AM tissue in the physiologic solution mixture, to thereby provide a therapeutic material for application to diseased tissue. The material may be fluid or formed from the lysates separated from the fluid.

The invention is still further, briefly, a three-dimensional (3D) device for treatment of diseased tissues by application of the 3D device to such diseased tissues, the 3D device comprising at least two sheets of dehydrated intact AM tissue. The stack of at least two sheets of intact dehydrated intact AM tissue is of sufficient thickness to be further formed to a preselected 3D shape for a specific therapeutic application.

All embodiments of the invention can be formed with or without honey, with or without preservatives, with or without additional cells and with or without additional chemicals.

### BRIEF DESCRIPTION OF THE DRAWINGS

The foregoing and other objects, features and advantages of the invention will be apparent from the following more particular description of preferred embodiments of the invention, as illustrated in the accompanying drawings; wherein

FIG. 1 is a schematic illustration of the general method of making a therapeutic material in keeping with the present invention;

FIG. 2 is an enlarged sectional illustration of a multi-layered device in keeping with the invention; and

FIG. 3 is an exploded illustration of one example of use of the 3D therapeutic device of the present invention.

Throughout the drawings like parts will be indicated by like numbers. Not all elements are shown in all figures. The drawings are not necessarily to scale, emphasis instead being placed upon illustrating the principles of the invention.

### DETAILED DESCRIPTION OF THE PREFERRED EMBODIMENT

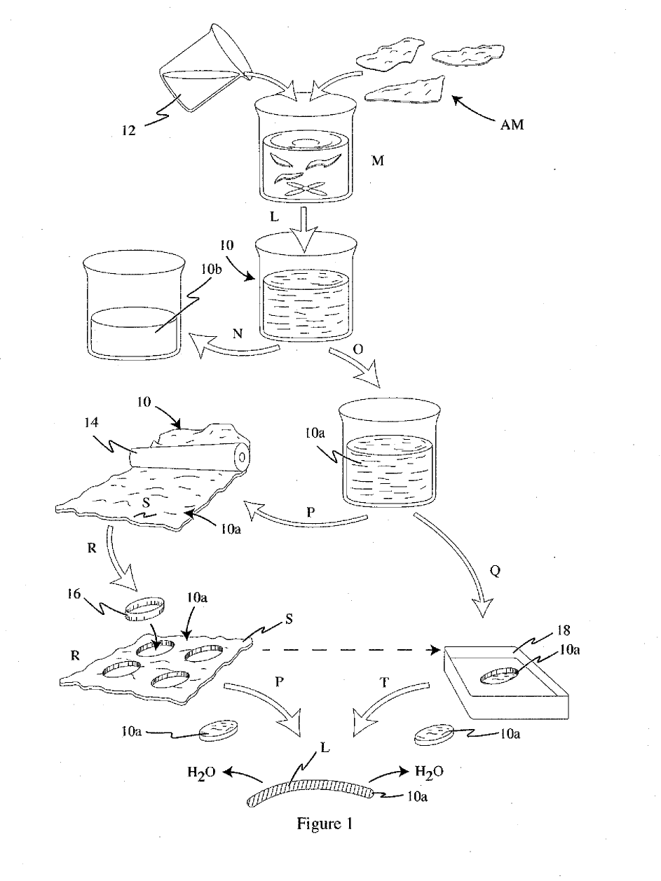

With reference to the drawings and as an introduction for better understanding of the following detailed description of the invention, FIG. 1 illustrates the basic steps of a method of making a therapeutic material, generally designated 10, in keeping with the present invention. The therapeutic material 10 can have a semi-solid/solid form, as indicated as examples by 10a, or the material can have a fluid form, as indicated at 10b. The illustrated device 10a is shown in FIG. 1 in its 3D form, in this case, in its final form of a contact lens, at T. A sheet S formed by a roll-press or other means of pressing micronized filtered AM lysate in Step P of micronized and processed AM lysates. Once the thick pad of filtered lysates are pressed, then in Step R the thick pad is cut up into useful size, as with a tiny cookie cutter, or otherwise, in Step S the portion cut out of the pressed sheet of AM the sheet can be shaped by a number of known means (molding, pressing, carving, etc.). Step Q illustrates semi-solid therapeutic material 10a being shaped by a mold, rather than by pressing, as in step P.

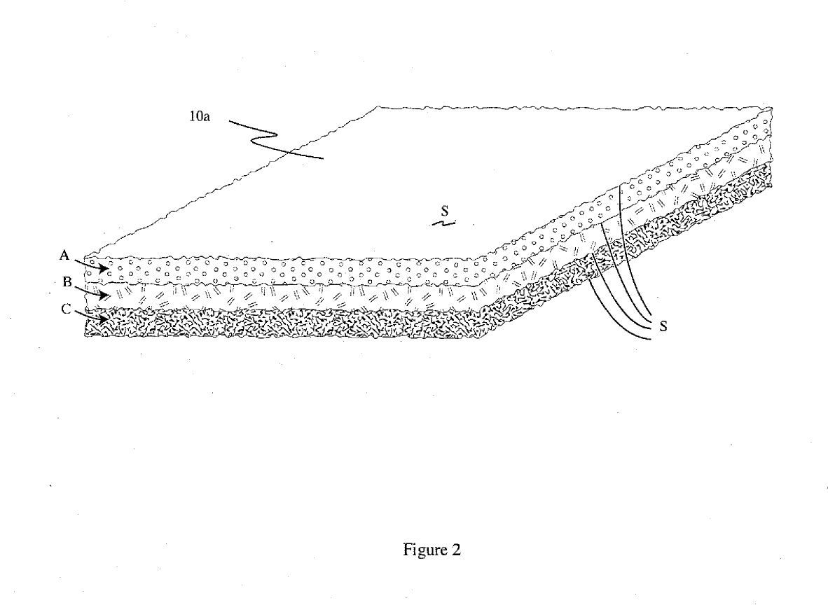

FIG. 2 shows an enlarged example of device 10a, in a more preliminary state, as a stack/block of laminated layers of AM, with the layers shown exaggerated, for purposes of illustration. This example would appear substantially the same whether the four sheets of AM are formed of intact sheets of AM or pressed lysate AM, later described. Device 10a in this example has four plies or sheets S of AM, each pair of adjacent sheets S being separated by a layer of another chemical/substance. The number of sheets of AM used in a particular 3D therapeutic device can vary as widely as necessary for an individual wound or other diseased tissue site for treatment therewith. The individual layers between adjacent AM sheets can be all of the same connecting substance, or each connecting layer can be of a different substance. The final composition of the device 10a will vary depending upon the specific intended use, the application site and the physician's preferences. Further details regarding the AM sheets and layers there between will be discussed later in this description.

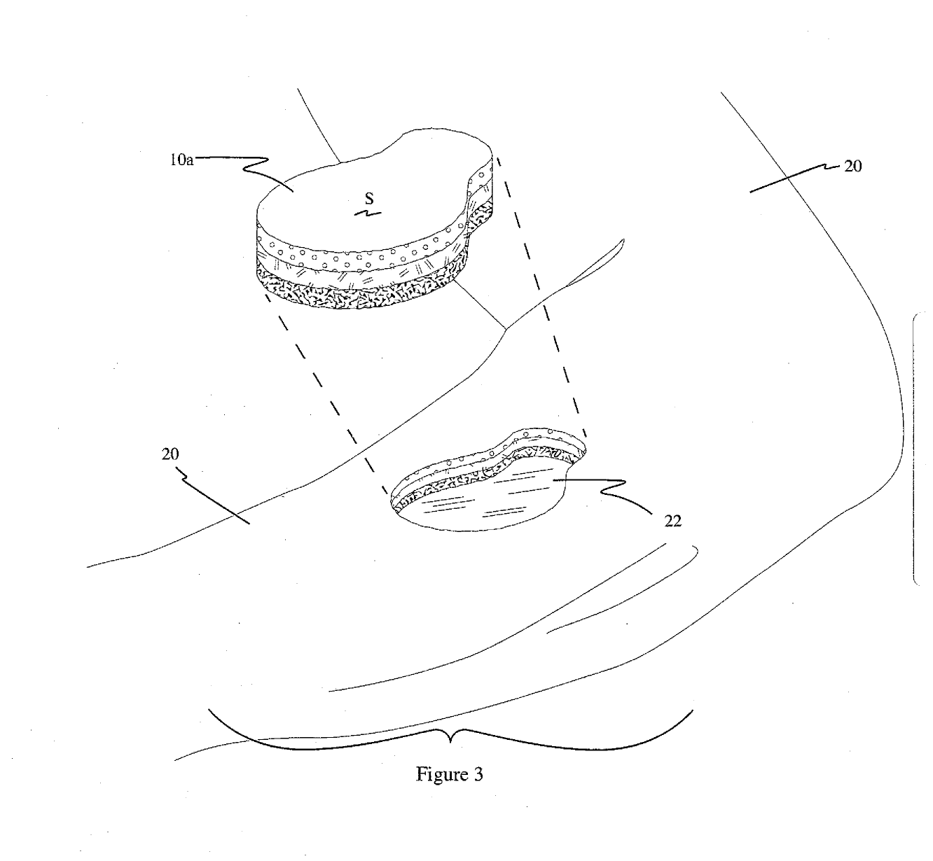

FIG. 3 shows an example of a potential use of an embodiment of the device 10a, formed to a customized shape and size for insertion into the patient's arm wound to enhance healing thereof. FIG. 3 clearly indicates how the specific shape of a skin wound W, such as might occur from a burn, cut or some other accidental injury or removal of a skin cancer, for example, can dictate the shape and depth of the needed 3D therapeutic device. In this illustrated example, the patient's arm A, has a wound W, which has been prepared to receive the custom-shaped device 10a. Once the device 10a has been inserted into the wound, device 10a can be secured there by stitching, gluing or otherwise sealing the device in place. Protection of the wound site W, after placement of the device 10a, can be handled with a protective bandage and cared for in keeping with current best practices of wound protection.

### Definition of “AM:”

Amniotic fluid and amniotic tissue includes cells that may perform some role in tissue growth and/or healing. This is true also of the chorionic membranes and the umbilical cord tissue. Because these various tissues each include multiple cell types and layers, any and/or all of which are known to have beneficial physiological properties, for purposes of clarity of the following description and claims, the term Amniotic Membrane(s) (“AM”) will be used throughout and is specifically intended to mean and include any and/or all of the above specified tissues, used individually or together, whether technically from the amniotic sac, chorionic, placenta or the umbilical cord, unless one of these other, non-amniotic terms is specified.

### Definition of “Micronized”:

It is to be understood for purposes of this document, including this description and the claims included, that “micronized” is intended to mean the process of reducing AM membranes/tissues into particles of a wide variety of sizes, readily visible, barely visible, and/or microscopic, which may or may not be limited to some given size range, depending upon the particular device or fluid being formed for therapeutic use. Thus, for simplicity and clarity of reading and understanding this document, and particularly the Detailed Description and Claims, “micronized,” as used herein, includes all useful means by which the AM tissue can be converted from a very large, thick, strong-walled bag by maceration, morselization, and most other terms referring to creating bits or small pieces of tissue, but specifically excluding fines which are so small and delicate that when dry they create a powdery substance which can be difficult to manage. Thus, for these purposes a consistency of baking flour, for example, would not be considered to be within the present meaning of “micronized.”

More specifically, the micronization of AM herein can be performed by any suitable known means, or even some means not yet fully developed or conventionally used for this purpose. Thus micronization may be performed by, but is not limited to methods including: manually or automatically grinding, slicing, pounding, chopping, cutting with lasers, blades, or sonic waves.

### The Method:

With reference to FIG. 1, production of a solid 3D therapeutic device 10a (a contact lens is used in this example) is illustrated. Device 10a is shown as being made from a manipulable, paste-like mixture of micronized AM lysates. The AM for the present invention is initially screened for infectious diseases and harvested in standard fashion. In the first Step M, the screened/harvested AM tissue is micronized in physiologic solution, all in a sterile laboratory environment. Once created, the AM suspension is filtered, at Step N, so that fluid 10b is separated from the tissue, and as one example only, contains no particulates any larger than approximately 22-microns. The 22-micron particle size is not absolute, but is included in this discussion as a useful example, because this is the size filter that is used for ASED preparation, previously discussed. The filter pore size could be larger or smaller, and is not limiting for this invention. The resulting filtered fluid 10b is retained for the next aspect of this invention that will be described in this patent; this valuable fluid is not wasted and its production is a vital step in the creation of AM-derived complex therapeutic fluids.

The filtered tissue is then, via Step O, passed into molds that create a form that is substantially the same proportionate shape, size, and volume as the recipient diseased tissue that will receive it, only somewhat larger in all dimensions, since the final solid form will become smaller after the molded material is lyophilized or otherwise dehydrated.

In the example, the mold is sized to emulate a collagen shield or conventional hydrogel bandage contact lens. Once molded, the form can be, at Step P, freeze-dried and packaged for shipment and storage in a sterile manner. In use, at Step T, the medical practitioner opens the packaging that contains the freeze-dried 3D solid form and either deploys it directly to the target diseased tissue, or alternatively re-hydrates the 3D solid form with any clinically advantageous therapeutic fluid (serum, plasma, physiologic saline, pharmaceutical antibiotics/anti-inflammatory agents, or honey derivatives or solutions), and then deploys the device to the target tissue.

For purposes of the present discussion and uses of the term in this document, “honey derivative” shall be understood as being any honey (NPA and/or PA) in any state of hydration, from anhydrous to any degree of water content. Native honey is generally about 17-18% water, but a honey “derivative” can be a solution of honey with 5% or 22% water, as examples only; i.e. native honey in which the water content may have been altered. Thus, in the claims which follow it is intended that use of the word “honey” shall include “honey derivatives.”

If the 3D solid form made by the method above is a contact lens, after the device is placed upon the ocular surface, the eye will typically be patched for temporary protection. An AM contact lens thus produced and deployed will subsequently dissolve over the following 8-24 hours. While in place, this device will not only elute the clinically advantageous therapeutic fluid(s) that were used to hydrate it, but also all of the biologically valuable chemicals and cells that are inherent in AM tissues, while simultaneously acting as a physical support for wound healing. After dissolution of the AM, the treatment can continue without the AM contact lens; or, if necessary, another, fresh AM contact lens can be prepared and deployed to the same site.

A solid 3D “contact lens shaped” AM of this new configuration will not require a rigid solid plastic ring to hold it in the proper use position, as described in the art; i.e., it will not fold when deployed, because the pre-formed shape of the therapeutic device and the globe of the afflicted eye substantially match. Further, in contrast to the known intact AM sheet eye treatments, there will be no foreign bodies placed on top of the new 3D AM device in its deployed, use position. The use of a standard hydrogel or silicone contact lens on top of an AM sheet causes reduced oxygen tension of the tissue, limits the access of tear-based nutrition and tear-based waste removal from the afflicted eye, and also provides a surface for the formation of potentially dangerous biofilms/infections. This risk is also eliminated by use of the new sterile contact lens-shaped AM device formed as described herein because the device 10a is deployed on the globe of the eye, under the eyelid, and then potentially also under a patch.

As only one useful example of an improvement provided by the present invention, a match in the shape of contact surfaces of the AM and the globe of the eye is more clinically and therapeutically desirable. This new AM contact lens strategy is more comfortable for the patient than providing the AM on the known rigid plastic carrier ring.

There are many suitable chemicals that can be used to fuse two or more layers of AM of either type, i.e., intact sheets of AM, or AM sheets formed of micronized lysates according to the invention. These chemical substances include honey, cross-linked or non-cross-linked chondroitin sulfate, cross-linked or non-cross linked hyaluronic acid, and amniotic membrane lysates, which are all examples of suitable substances for the union of separate intact sheets of amniotic membrane before final lyophilization.

Honey is one especially preferred substance for use particularly in combination with AM for tissue healing because of the important opportunity for synergistic effects of the intrinsic healing properties of both the honey and the AM, as is discussed further hereafter. For example, in the device 10a illustrated in FIG. 2, layer A can include an antibiotic, layer B can be honey, and layer C can be cross-linked hyaluronic acid, if that order and combination is what most benefits the particular diseased/injured tissue being treated with device 10a. For the wound application depicted in FIG. 3, the physician might prefer, instead, to make the all three layers A, B and C, (or more) with honey.

This embodiment of the new 3D therapeutic device made from AM lysates, when formed as described above for use on the eye, retains all of the benefits of prior AM preparations for the ocular surface, but acquires the advantage, of potentially dissolving over a shorter time course than can a complete intact sheet of AM. If necessary, however, a 3D therapeutic device in keeping with the invention can be formed as a contact lens shaped element that will last much longer. Breakdown of device 10a and elution of any cells or chemicals included in a specific construction thereof can be controlled by the producer on a customized basis, as is suitable for a specific type of therapeutic use. Alternatively, the laminated AM embodiments of this invention are designed to last much longer once they are deployed to the diseased tissue. A clinical setting where a longer duration embodiment would be desirable is a recalcitrant non-healing infectious or autoimmune based corneal ulcer or “melt.” In this clinical setting, a laminated AM preparation shaped as a contact lens would be ideal. Although single sheets of AM may be more advantageous for clinical conditions that require 3-5 days of AM contact, the present embodiment that is formed from AM lysates is ideal for applications at the completion of intraocular surgery, (like cataract surgery, for example only), where the absence of the contact lens much sooner, i.e. the next day after the procedure, is desirable.

The described contact lens-shaped embodiment of the invention is also more desirable than a conventional porcine-derived collagen shield, because the tissue origin is human, and AM has many advantageous cells and chemicals that are inherent to AM that are not inherent in the tissues from which collagen shields are derived. Specific cell types that are not present in AM include peripheral blood cells and bone marrow cells, non-AM stem cells, structural cells (bone, skin, hair, cartilage, tooth), and immune effector cells both in their inactive states and after stimulation by antigens including T-cells, B-cells, natural killer cells, macrophages, and plasma cells. All embodiments of the present invention are suitable for selective addition of any of the above cell types when such addition is called for in treatment of a particular diseased tissue, as determined by the practitioner.

A further embodiment of the present invention is a method whereby the filtered amniotic membrane lysate particles can be manually or mechanically automatically rolled out in a thin flat sheet, as indicated at step R in FIG. 1 and then fenestrated or punctured, in many locations to facilitate the dehydration/lyophilization process, and to facilitate the dissolution of the resulting device and any chemicals or specialized cells therein, once it is placed on, or in, a human or animal's body. Alternatively, the roll-pressed thin sheet of amniotic membrane lysates may be kept initially planar, but non-fenestrated. The sheet is then stamped in a circle the size of a contact lens (step S in FIG. 1), and formed (step T) in or on a mold. The mold can be smooth, to form the lens surface shape to be uninterrupted; or in an alternative embodiment, the mold per se can have various protuberances that create fenestrations in the stamped circle sheet of filtered amniotic membrane lysates. This will accomplish the same goals as fenestrating a roll of pressed lysates before molding, and might make handling easier and with less wastage, although both methods and certainly others are within the scope of the present invention and will be apparent to one skilled in the art. Similar methods of formation can be used to create other shapes of the new device, the preferred choice of method varying to some extent with the size and shape of the desired end product. When a 3D AM device of the invention is needed for an unusual purpose, the described methods herein permit the required device to be completely customizable for essentially any size or shape needed.

It is understood that other useful methods of forming the lysates end product are also within the scope of the invention although not specifically described herein, as they are too numerous, but will be apparent to one skilled in the art. In some instances it may even be suitable that the therapeutic device of the invention can be formed by hand molding.

### Intact AM Sheets:

In a further embodiment of the invention, as opposed to molding lysates of amniotic membrane, the same objective can be met by first bonding two or more layers of intact sheets of amniotic membrane, and then molding, press forming, cutting, carving, or altering the resulting multi-ply block of amniotic membrane into any desirable therapeutically beneficial 3D shape. Just as with a therapeutic device formed of micronized AM lysates, the materials used to bond or unite individual sheets of intact amniotic membranes to create a multi-ply block for further manipulation can be anything that would create a suitable bond and which are biocompatible. This includes such chemicals as honey, cross-linked or non-cross-linked chondroitin sulfate, cross-linked or non-cross-linked hyaluronic acid, and lysates of amniotic membranes. There are many more biocompatible bonding agents, and this list is not limiting for this invention. Laminates made from intact sheets of AM will be structurally stronger than the same laminated structures made from sheets formed from AM lysates. Clinical and surgical settings that would benefit from stronger material include abdominal wall/hernia repair, pelvic floor reconstruction, skin defects under tension, and colon and rectal prolapse repair, for example only.

Another advantage of the invention described herein is the opportunity to modify the character of the AM product per se, before it is lyophilized, or otherwise dehydrated. For example, honey or chemical honey derivatives can be incorporated onto solid sheets of AM or directly into lysates mixtures before, during, or after molding into the final 3D form. The addition of honey or honey derivatives to the AM product will alter the rate of dissolution of the AM while simultaneously conferring the clinically therapeutic benefits inherent in honey and the 3D structure developed according to the invention elute into and around the wound or diseased tissue. The types and advantages of various honeys have been discussed above, in the Background of this document, in order to indicate that there are different types of honeys to select from, with different characteristics, that will be very helpful to understand in making the selection of which honey to use in a given therapeutic device or fluid in keeping with the invention.

In further embodiments of the present invention other chemicals are added either to intact sheets of AM or to lysates of AM before, during, or after molding either form of the AM into a 3D device for treatment of diseased tissue. Examples of such chemical additives are provided below. The chemicals can be heated before applying them to intact sheets of AM, or mixed with lysates thereof, if heating facilitates the process of the particular chemicals becoming intimately affixed to or homogeneously entrapped in the matrix of the AM lysates. The added chemicals will controllably alter the rate of AM dissolution after placement of the AM device on the target diseased tissue, thereby offering the clinician new and valuable alternatives to any of the intact sheet AM products that are now available.

As examples only, hyaluronic acid (HA) and/or chondroitin sulfate can be applied to an intact sheet of AM or the described lysates thereof. These chemical macromolecules become intrinsic constituents of the AM device. The HA and/or chondroitin sulfate are important contributors to tissue form and shape and are instrumental to wound healing. When hydrated, in their native unaltered states, they are gels, but when dehydrated, they become solids. Alternatively, these glycosaminoglycans can be cross-linked, which renders them more resistant to degradation by tissue enzymes (like hyaluronidase, for example only) designed to break down and clear these molecules from tissues. Cross-linked HA and cross-linked chondroitin sulfate that are incorporated into any AM preparation would therefore be expected to prolong the dissolution rate of the AM preparation more than if unaltered HA and/or chondroitin sulfate were similarly incorporated. The addition of HA and/or chondroitin sulfate, cross-linked or not cross-linked, can occur before, during, or after the AM lysates are molded and lyophilized, or otherwise dehydrated. They can also be incorporated in liquid or powder form after the molded AM lysates are dehydrated.

If liquid HA is incorporated into molded and dried AM forms, the product would need to be dehydrated a second time, and the HA would thus strictly be located only on the surface of the formed device. Likewise, HA can be incorporated at any stage in the processing of intact AM sheets to confer to them variable rates of dissolution once placed on the target tissue. The ability to modify the dissolution rate of an AM preparation offers a novel way to offer the clinician new opportunities for more controlled therapeutic interventions. The use of a chemical like HA is analogous to the use of honey, described below. The difference is not in the manner that the chemical is handled during the production of these devices, rather it is the intrinsic character of the chemical that is incorporated that confers the various therapeutic clinical advantages to the final AM product. Thus, the practitioner can select the specific content of the therapeutic device, depending upon the particular need; i.e. what type of tissue is to be treated and for what specific ailment.

The amniotic membrane (AM) is an avascular tissue that forms the innermost layer of the fetal membranes. It is composed of five layers: an epithelial cell monolayer, an acellular basement membrane layer, a compact layer, a mesenchymal cell layer, and a spongy layer placed in close proximity to the chorion.

Two types of cells have been isolated from the AM. They are human amniotic epithelial cells (HAECs) and human amniotic mesenchymal stromal cells (HAMSCs). Both types of cells possess stem cell characteristics, differentiation potential toward lineages of different germ layers, and immunomodulatory properties. These cells do have surface antigens which cause them to have the potential to induce inflammation. Because these cells have real potential therapeutic benefit when used to treat disease, some therapeutic amniotic membrane preparations retain the therapeutic cells as a part of the final deployed product. Other indications for amniotic membrane therapy benefit from treatments that do not provoke inflammation. For those applications of amniotic membrane therapy, these intrinsic AM cells can be removed by a number of standard methods well known in the field.

Because the amniotic membrane has five distinct layers in a specific natural orientation, it is important to contemplate the orientation of intact sheets of amniotic membranes, when they are used on or in the body. All body surfaces that are populated by layers of cells rely on a material known as basement membrane to support, secure, and orient adherent cell layers. A layer of cells will not remain intact in or on the body if it is not properly attached to a basement membrane Likewise, a body surface that becomes injured, like the cornea for example only, will not allow a proper cell layer to heal over a structural defect unless there is a sufficient basement membrane underlying the cell layer that is attempting to resurface the injured territory. Normally, cells have the capacity to create their own basement membranes, but this is metabolically stressful, and it slows down healing and sometime creates a deranged conformation of the resulting healed structure; the basement membrane created in adverse settings can be less than idea.

Amniotic membranes have a distinct basement membrane layer. This is the intact layer to which the epithelial cells are attached. For applications of amniotic membrane where it is used as a therapy to promote the healing of a body surface, it is more effective to orient the intact sheet of amniotic membrane so that the basement membrane surface is facing the surface that will receive an advancing cell layer that is trying to grow over the defect. An intact and healthy sheet of amniotic membrane will greatly facilitate a more rapid re-surfacing of the intended cell layer, as opposed to causing the cell layer to create a proper basement membrane de novo. This feature of AM intact tissue is utilized in various versions of the present device, in which the device is formed of multilayered intact sheets of AM, rather than the described sheets or other forms of micronized AM lysates, as will be described further, later herein.

### The 3D Therapeutic Device:

Accordingly, in any embodiment of the present invention in device form where intact sheets of amniotic membrane are used for a therapeutic purpose, the orientation of the sheet is important to maximize the effectiveness of the device. This applies to single sheets of amniotic membranes, laminates, preparations that are hydrated or dehydrated, and those which have had other chemicals applied to either outermost or innermost surface of intact sheets. In this sense, all embodiments of intact sheets of amniotic membrane have a tissue-based preferred directional orientation.

Likewise, if different chemicals are applied to the opposing sides of an intact sheet of amniotic membrane, this will confer an additional optimal orientation that is chemical-based. For example only, if a single intact sheet of amniotic membrane has one side coated with an antibiotic and the opposite side coated with hyaluronic acid, then it may be preferable to place this device with the antibiotic coated surface closest to the surface of the treated diseased organ, such as described with reference to FIG. 3.

With micronized AM lysates sheets, which are substantially homogenous after production of the 3D therapeutic device 10a, there is no definite orientation. Once amniotic membrane of the present invention has undergone micronization to make intact sheets smaller (through crushing, cutting, blending, grinding, milling, and other standard methods), the tissue-based orientation is no longer germane, as the resulting population of amniotic membrane pieces have had their tissue-based orientation randomized. However, in any embodiment of the present invention where these amniotic membrane pieces are used as building blocks to mold purposeful 3D structures, the resulting forms will retain a chemical-based required orientation if the manufacture of the molded forms places different chemicals onto different surfaces of the final form. Even a structure that is substantially planar that is made from molded small pieces of amniotic membranes, either a single intact sheet or multi-ply, laminated structure, will lose its tissue-based orientation, but retain its chemical-based orientation, if different chemicals are applied to the top and bottom micronized lysates surfaces.

Other embodiments of AM lysates are those that are prepared in a 3D shape that is amorphous. During the creation of AM lysates, just before the step where the amorphous AM lysate paste is formed into specific discrete 3D forms, the lysates can be partially dehydrated, sterilized, and packaged for distribution for use as a paste. This AM paste can be deployed by the end user as a tissue putty that contains all of the physical-chemical advantages of dehydrated 3D forms, but offers the advantage of being customizable to the 3D contours of the intended body part at the time of deployment. This AM lysate paste can be used to coat a tissue surface or be used to fill any cavity formed by trauma or surgery. Likewise, the AM lysate paste can be dehydrated in a 3D shape that amorphous. The fully dehydrated AM lysate paste results in a material that also has an amorphous 3D shape that behaves like granules, analogous in structure and handling to sand or table salt, but not like baking flour. This product can be deployed in an analogous manner as AM lysate putty if it is rehydrated, or as a dry material that can be used in similar clinical settings as AM paste, but where there are no suitable rehydrating solutions available, or where a dry preparation is more desirable. An example of a clinical setting where the dry granules would be desirable is an actively bleeding penetrating battlefield wound, where granules manufactured in conjunction with antibiotics and thrombin can be packed into the wound to stabilize the patient, limit the risk of future infections, and limit clinically relevant blood loss.

The foregoing discussion would largely apply to embodiments of the present invention that are derived from chorionic membranes. The chorion likewise has a basement membrane and tissue-based polarity.

### The Therapeutic Fluid:

Another embodiment of the present invention is a therapeutic fluid 10b, indicated at Step N in FIG. 1, and derived from the filtered fluid/supernatant that is separated from the micronized AM tissue. Preferably, and in order to create a significant volume, the AM fluid 10b is created from pooled AM tissue donors. The advantage of this therapeutic fluid 10b is that it can be prepared in bulk, as opposed to the known ASEDs previously discussed. Therapeutic fluid 10b that is separated from the micronized AM contains a plethora of chemicals that would be impractical or impossible to study as individual constituents in an eye drop preparation that was “built” from water and a formula of components. However, fluid 10b, once separated from the micronized AM, can be diluted with physiologic solution prior to use. A physician can prescribe a preferred dilution, and the manufacturing company can also provide a variety of concentrations. This fluid product is biological in nature, and thus should be frozen, lyophilized, or inclusive of a preservative upon completion of production, to extend the shelf life of the fluid.

The therapeutic fluid 10b, just described, can be an eye drop in one embodiment. The eye drop thus created is a very effective treatment for patients with dry eyes. The dry eye market is considerable, with United States sales of Restasis® drops alone exceeding one billion dollars per year. Another embodiment of therapeutic fluid 10b features the incorporation of honey, which confers clinically therapeutic synergistic benefits to the product, both chemically and physio-chemically for product retention once deployed on the ocular surface. One aspect of honey that enhances the use of eye drops is the increase in viscosity that it provides to an aqueous solution. This is especially useful for people with chronic dry eyes.

Likewise, a therapeutic fluid derived from the methods described above, with or without the incorporation of other chemicals, such as HA or honey, can be deployed on any other body surface, on devitalized skin, for example. This therapeutic fluid can also be co-packaged with lyophilized solid AM preparations and used to hydrate the solid before deployment on the target tissue. Formal clinical trials are expected to be required for some of the new products made in keeping with the invention. Moreover, some of the products made according to the invention will likely be classified and regulated by the FDA as drugs; products of this kind are remarkably safe and effective, and should be well received in the market place.

Another embodiment of the present invention is a therapeutic fluid, such as an eye drop, for example, that is an admixture of the amniotic membrane tissue-derived therapeutic fluid described above, with a honey-derived aqueous solution that contains either (Non-Peroxide Activity) NPA and/or (Peroxide Activity) PA honey, in any aqueous dilution from 1% to 99%, with or without preservatives to prevent fungal and/or bacterial growth. The admixture of these two biologically derived products will impart the final chemical solution with desirable properties that are present in both products, independent of one another, but will also provide synergistic therapeutic clinical benefits. The incorporation of honey with any of the amniotic membrane-derived products described will extend the shelf life of the resulting combination product because of the intrinsic antibiosis properties of honey.

Another advantage of the incorporation of honey or honey derivatives into the admixture with AM products is its intrinsic high viscosity, which permits the resulting therapeutic fluid to have a greater residence time when applied topically to the body and thereby allows for a more prolonged therapeutic effect. Additionally, the honey will offer the admixture intrinsic antibiosis properties, with or without the addition of traditional preservatives. The admixture of a honey-derived therapeutic fluid with an amniotic membrane tissue-derived therapeutic fluid can be created using the fluid recovered from 1) completely filtered micronized AM lysates, in which case the resulting product is a pure liquid, and may potentially be characterized and regulated as a drug, or 2) a honey mixture combined with lysates of micronized amniotic membrane that are only partially filtered, and retain some particulate character, and as such, may be characterized and regulated as a device.

A further embodiment of the present invention is the admixture of NPA and/or PA honey, in any aqueous dilution (1%-99%) that is added to the micronized AM lysates, so that when the resulting paste is dehydrated in the form of a contact lens or any other therapeutically useful 3D form, the honey component is mixed uniformly throughout the molded device and into the dehydrated end-product.

Alternatively, in another embodiment, a device that is solely derived from the amniotic membrane lysates can then be dipped in or otherwise coated with either an NPA or PA honey solution, or an admixture of the two types of honey (aqueous dilution, 1%-99%) and then dehydrated onto the outside of the amniotic membrane-derived contact lens or any other 3D form, to provide a coating to the formed device. With this product the honey is preferably dehydrated onto the surface of the formed amniotic membrane lysates device during production and then the device can be packaged for storage or shipping in a dehydrated state. The above honey-dipped version of device 10a can also be produced with other substances mixed into the micronized AM lysates paste.

After any of the AM embodiments are manufactured to their final therapeutic 3D form, they can also be populated with many different kinds of viable therapeutic cells. If the AM 3D therapeutic form is not dehydrated, the cells can populate the device before it is shipped to the practitioner in its hydrated or partially hydrated state. If the device requires dehydration, then the dehydration step must take place first, before the cells are incorporated, as dehydration will cause any incorporated cells to become non-viable. Many cell types can be contemplated as therapeutically beneficial for incorporation into these 3D AM devices, and include any type of class of circulating peripheral blood cells, various types and classes of bone marrow cells, stem cells of any kind, structural cells (bone, skin, hair, cartilage, tooth), immune effector cells both in their inactive states and after stimulation by antigens inside or outside the body where they are deployed, these latter include T-cells, B-cells, natural killer cells, macrophages, and plasma cells.

An additional embodiment of the present invention is the application of honey powder (anhydrous honey with 0% water content) as a coating to the surface of any previously dehydrated amniotic membrane preparation irrespective of whether is it intended for application for a human or other animal, or whether it is intended for topical or internal use. This is distinct and different from the embodiment described above, where an amniotic membrane is first dehydrated, then dipped in honey, and then dehydrated a second time, to bind the honey to the surface of the amniotic membrane.

Alternative packaging and shipping models include: 1) for the 3D molded amniotic membrane-derived product or prepared intact AM sheet can be packaged in a solution of honey (likely in the minimally hydrated state, 1-18% water), so that the amniotic membrane-derived device comes in a partially hydrated state, as opposed to the other embodiments which are packaged in their fully dehydrated state and must be hydrated with saline, an antibiotic, a non-steroidal anti-inflammatory, or a steroidal pharmaceutical before placement onto the surface of the body; and 2) the lyophilized AM product can be co-packaged in a kit with a suitable honey that is housed in a separate container. At the time of use, the practitioner opens the container of honey and uses it to hydrate and deploy the lyophilized solid AM to the target tissue.

An example of a therapeutic application for AM laminates would be in the battle field. Many injuries are penetrating in nature from rapidly moving objects, like bullets and shrapnel. An open wound has many potential adverse effects on the patient. Theses wounds are typically dirty and prone to infection. They are often bleeding. They are often structurally destabilizing to the injured body part. A thick laminated AM sheet can be contemplated to contain honey, antibiotics, and thrombin (a natural chemical substance made by the body that induces blood clotting.) In the field, a medic could open sterile packages of this material, and pack it into cavities that are opened by penetrating objects, to bolster their structure, deliver antibiotics and reduce blood loss. This will make transportation of an injured person more stabile, and the deployment of this device will reduce blood loss and the risk of infection. Many such devices can be opened and packed into these open wounds. Similarly, if the wound is not penetrating, but instead is a thermal injury or abrasion of the body surface, the same device can be used topically, with or without a dressing, to effect the same therapeutic benefits that deployment of the device has when used for penetrating injuries. A dehydrated AM laminate with antibiotics, honey, and thrombin, for example only, will have a very durable “shelf life” and can be expected to remain safe and effective in the theater of war. This is also true of the fully dehydrated AM lysate granule preparations.

The use of any of the honey-amniotic membrane-derived products that are substantially planar (curved or flat) may require hydration with some fluid just prior to placement, to ensure full pliability and to allow the product to absorb water from an applied solution, since the product will be very hygroscopic if applied directly in its purely dehydrated state. However, application of the highly hygroscopic substantially planar AM preparation is beneficial in that it will immediately draw fluid out from the diseased tissue to which it is applied, and will likely foster adherence of the membrane to the target tissue. This physical-chemical behavior, in some applications, may be used to the practitioner's advantage, so that hydrating the AM membrane before its therapeutic application to the body, externally or internally, may be intentionally avoided.

The present invention includes a method of preparation of AM that enables a manufacturer to create a 3D shape of solid AM that can be deployed topically or internally, and more closely match the contours and dimensions of the tissue that receives the AM. This will vastly expand the applications of this valuable treatment, as solid molded 3D AM products can be deployed on the skin, on the scalp, on the anus, on the introitus, or on the lips. Molded 3D AM products formed using these methods can also be deployed internally, in the mouth, along the gastrointestinal tract, along the lumens of the female reproductive tract, in joint spaces, and in surgery where tissue planes are cut, in any internal organ system in the body.

Moreover, there instances wherein the fluid form of the new therapeutic material described herein is useful, for example, as a lavage for flushing out a subcutaneous space that has the potential for infection or is already infected and/or, to facilitate healing of the wound. Deep puncture wounds, animal bite wounds and intra-peritoneal infections are examples of such useful applications.

The specific choice of chemical or other substance between any two sheets of AM (whether from lysates or intact AM sheet) can be selected by the practitioner depending upon the usage. It is anticipated that as usage of the present invention becomes common practice, certain combinations of frequently selected chemicals or other substances in certain positions within a device 10a may become standard products, whereas there can also remain the option for custom orders of the device as a therapeutic medical product. Likewise, the potential uses for the new therapeutic material in fluid form are wide and great. Certain formulations will likely become standards, with custom products being available from formulary pharmaceutical providers.

Likewise, the bonding of two or more intact (non-micronized) sheets of amniotic membranes to create a multi-ply/laminated stack of AM sheets will also permit the controlled formation of 3D forms that can be customized for a wide range of topical and internal therapeutic applications. Once formed, a block of this multi-ply material can be shaped by any method, including cutting, carving, molding, pressing, twisting, as examples, to achieve the final size and shape of the intended device. Thus, the new 3D therapeutic devices can be created by shaping the described “pasty or “doughy” lysates of micronized amniotic membrane, or by bonding intact multiple laminated, substantially planar sheets of either intact amniotic membranes or laminated sheets of micronized AM, and then shaping the resulting 3D structure afterwards.

As explained above, with the above invention at hand, it is not necessary for the medical practitioner to limit the use of AM intact sheets to small, thin sheets for limited applications, struggling with issues such as the susceptibility of the material to the slightest air movement and folding issues. Rather, as explained, sheets of amniotic membranes can be bonded to one another in a multi-ply fashion with a bonding agent and can be formed into a device of increased thickness, even a “block” of such amniotic membrane. This can be accomplished with intact AM sheets or with the micronized AM lysates rolled out, as explained. Once created, the block can be molded, press formed, cut, carved, or shaped by any useful method to create substantially any 3D shape desirable for therapeutic deployment to diseased tissue in or on a mammalian body. Examples of such useful shapes are an ophthalmic contact lens, skin patching, and even a device that can be placed in an articular joint. The possibilities are myriad, particularly when the basement membrane feature of intact amniotic membranes is considered.

This feature of intact AM sheets is especially useful for repair of damaged blood vessels or creation of new blood vessels. Rather than stacking multiple sheets of intact AM and then cutting out a shaped, for this embodiment of the invention at least two intact sheets of AM would be layered against each other; i.e., laminated, but with the basement membrane layer of each sheet specifically facing outwardly. The laminated intact sheets would then be formed into a tube shape such that in use a basement surface membrane faces either the outer surface of the tube or the inner luminal surface of the tube. This embodiment could be used to create new blood vessels.

This embodiment would be a laminate that is created to a thickness that approximates the thickness of a vascular wall that is rolled and sealed by any method that produces a laminated AM tube, with the basement membrane either on either the luminal side (so vascular endothelium can grow over it), the outside surface (so epithelial cells can grow on it), or on both surfaces. Due to the inherent polarity of the AM membrane, if the multiply laminate (for strength) is oriented with the inner most sheet so the basement membrane faces inwardly and the outer-most sheet faces outwardly, there results a structurally strong tube that has native intact basement membrane on all surfaces. A desirable use for such a structure is in vascular bypass surgery, such as coronary artery bypass surgery, where the surgeon must harvest an artery from another part of the body to serve as the new blood flow conduit. Larger tubes of this material can be formed for larger bypass operations, like a femoral-popliteal artery bypass, where tubes made of Gortex® have previously been used. If a two-ply laminate form of the intact, fused membranes is too thin for the particular application, multiple layers can be added, to achieve the necessary thickness, and arranged so that the two outermost layers are each positioned with the basement membrane facing outwardly.

The present compositions, articles, fluids, devices and/or methods disclosed and described above are not limited to specific methods unless otherwise specified, or to particular reagents unless otherwise specified, and as such may vary, as explained. It is also to be understood that the terminology as used herein is used only for the purpose of describing particular embodiments and is not intended to be limiting.

As various modifications could be made to the exemplary embodiments, as described above with reference to the corresponding illustrations, without departing from the scope of the invention, it is intended that all matter contained in the foregoing description and shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

What is claimed is:

1) A three-dimensional (3D) device for treatment of diseased tissues by application of the 3D device to such diseased tissues, the device being comprised of micronized amniotic membrane (AM) tissue in a physiologic solution and dehydrated to a paste-like consistency for selective manipulation into a specific preselected form.

2) The 3D device of claim 1, wherein the physiologic solution with the micronized amniotic membrane tissue comprises an aqueous mixture of honey.

3) The 3D device of claim 2, wherein the honey in the aqueous mixture of honey is selected from the group consisting of Non-Peroxide Activity (NPA) honey, Peroxide Activity (PA) honey and an admixture of PA honey and NPA honey.

4) The 3D device of claim 2, wherein the aqueous mixture of honey further comprises a chemical preservative.

5) The 3D device of claim 1, wherein the 3D device is molded into a specific preselected form.

6) The 3D device of claim 5, wherein the 3D device is pressed into a sheet.

7) The 3D device of claim 6, wherein the sheet has a selectively variable thickness.

8) The 3D device of claim 5, wherein the 3D device further comprises honey.

9) The 3D device of claim 6, wherein the 3D device is formed of a stack of at least two sheets of the micronized AM tissue.

10) The 3D device of claim 9, wherein the stack of at least two sheets of micronized amniotic membrane tissue is of sufficient thickness to be further formed to a preselected 3D shape for a specific therapeutic application.

11) The 3D device of claim 9, and further comprising an adhesive substance between adjacent sheets of the at least two sheets of micronized AM tissue.

12) The 3D device of claim 11, wherein the adhesive substance between any two adjacent sheets of the at least two sheets of micronized AM tissue is comprised of honey.

13) The 3D device of claim 8, the 3D device comprising at least one sheet of micronized AM tissue, wherein the at least one sheet is dehydrated, coated with honey and dehydrated again, so that the at least one sheet of micronized AM tissue is coated with anhydrous honey powder, wherein the honey is selected from the group consisting of NPA honey, PA honey and an admixture of PA honey and NPA honey.

14) The 3D device of claim 13, wherein the honey further comprises a chemical preservative.

15) The 3D device of claim 13, wherein the 3D device is formed of a stack of at least two sheets of micronized AM tissue.

16) The 3D device of claim 15, wherein the stack of at least two sheets of micronized amniotic membrane tissue is of sufficient thickness to be further formed to a preselected 3D shape for a specific therapeutic application.

17) The 3D device of claim 16, wherein the 3D device is formed into a specific preselected form.

18) The 3D device of claim 17, wherein the specific preselected form is a therapeutic, ocular contact lens.

19) The 3D device of claim 16, and further comprising an adhesive substance between adjacent sheets of the at least two sheets of micronized AM tissue.

20) The 3D device of claim 17, wherein the adhesive substance between any two adjacent sheets of the at least two sheets of micronized AM tissue is comprised of honey.

21) The 3D device of claim 13, wherein the at least one sheet of micronized AM tissue is marked to indicate intended direction of orientation of the 3D device during application to the diseased tissue.

22) The 3D device of claim 1, wherein the 3D device is further dehydrated after manipulation into a specific preselected form.

23) The 3D device of claim 22, wherein the 3D device is fenestrated to facilitate dehydration.

24) The 3D device of claim 22, wherein the 3D device is rehydratable for use.

25) The 3D device of claim 22, wherein the 3D device is rehydratable with a solution including at least one substance selected from the group consisting of honey, honey derivatives, pharmaceuticals, pharmaceutical antibiotics and anti-inflammatory drugs, hyaluronic acid, cross-linked hyaluronic acid, chondroitin sulfate, cross-linked chondroitin sulfate, and anti-inflammatory fluids filtered from micronized amniotic membrane lysates.

26) The 3D device of claim 22, wherein the 3D device is rehydratable with a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

27) The 3D device of claim 1, wherein the 3D device is an ocular contact lens.

28) The 3D device of claim 27, wherein the ocular contact lens has a concave surface for placement against the corneal portion of an eye globe and a convex surface for placement facing outwardly of eye when in therapeutic use position, and further wherein the ocular contact lens comprises a coating of a first substance on the concave surface and a coating of a second substance on the convex surface.

29) The 3D device of claim 1, wherein the 3D device is frozen until use.

30) The 3D device of claim 1, and further comprising a chemical preservative.

31) A therapeutic fluid for treatment of diseased tissues by application of the therapeutic fluid to such diseased tissues, the therapeutic fluid being formed from liquids generated and separated during micronization of amniotic membranes (AM).

32) The therapeutic fluid of claim 31, wherein the therapeutic fluid is substantially free of particulate matter.

33) The therapeutic fluid of claim 31, wherein the therapeutic fluid formed from liquids generated and separated during micronization of AM also comprises an aqueous mixture of honey.

34) The therapeutic fluid of claim 33, wherein the honey in the aqueous mixture of honey is selected from the group consisting of NPA honey, PA honey, and an admixture of NPA honey and PA honey.

35) The therapeutic fluid of claim 31, and further comprising a chemical preservative.

36) The therapeutic fluid of claim 31, and further comprising a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

37) The therapeutic fluid of claim 31, wherein the therapeutic fluid is dehydratable to facilitate shipping and storage.

38) The therapeutic fluid of claim 37, wherein the dehydrated therapeutic fluid is rehydratable for use.

39) The therapeutic fluid of claim 38, wherein the dehydrated therapeutic fluid is rehydratable with a solution including at least one substance selected from the group consisting of honey, honey derivatives, pharmaceuticals, antibiotics, hyaluronic acid, cross-linked hyaluronic acid, chondroitin sulfate, cross-linked chondroitin sulfate and anti-inflammatory fluids filtered from micronized amniotic membrane lysates.

40) The therapeutic fluid of claim 31, wherein the therapeutic fluid is frozen until use.

41) The therapeutic fluid of claim 33, wherein the therapeutic fluid is eye drops.

42) A method of making a therapeutic material for treatment of diseased tissues comprising the steps of:
a) providing amniotic membrane (AM) tissue,
b) providing a physiologic solution,
c) mixing the AM tissue and the physiologic solution,
d) micronizing the AM tissue in the physiologic solution mixture, to thereby provide a therapeutic material for application to diseased tissue.

43) The method of claim 42, wherein the physiologic solution is an aqueous solution of honey, the honey being selected from the group consisting of Non-Peroxide Activity (NPA) honey, Peroxide Activity (PA) honey and an admixture of NPA honey and PA honey.

44) The method of claim 42, and further comprising e) substantially separating the micronized AM tissue lysates from the supernatant fluid of the micronizing mixture.

45) The method of claim 44, and using the supernatant fluid separated in step e) as a therapeutic fluid for treatment of diseased tissues.

46) The method of claim 45, and further comprising adding a chemical to the therapeutic fluid, the chemical comprising a substance selected from the group consisting of honey, honey derivatives, pharmaceuticals, pharmaceutical antibiotics and anti-inflammatory drugs, hyaluronic acid, cross-lined hyaluronic acid, chondroitin sulfate, cross-linked chondroitin sulfate, and anti-inflammatory fluids filtered from micronized amniotic membrane lysates.

47) The method of claim 45, and further comprising adding a solution of cells to the therapeutic fluid, the solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

48) The method of claim 44, and further comprising the step of combining the aqueous mixture of honey with the micronized AM tissue during step c) to a concentration such that the resultant combination is of sufficiently low viscosity as to permit the therapeutic material formed therewith to be used as a therapeutic fluid to be applied to diseased tissue.

49) The method of claim 48, and further comprising adding a chemical to the therapeutic fluid, the chemical comprising a substance selected from the group consisting of honey, honey derivatives, pharmaceuticals, pharmaceutical antibiotics and anti-inflammatory drugs, hyaluronic acid, cross-lined hyaluronic acid, chondroitin sulfate, cross-linked chondroitin sulfate, and anti-inflammatory fluids filtered from micronized amniotic membrane lysates.

50) The method of claim 48, and further comprising adding a solution of cells to the therapeutic fluid, the solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

51) The method of claim 45, and further comprising the step of adding honey to the therapeutic supernatant fluid separated from the AM lysates in step e), the honey being selected from the group consisting of PA honey, NPA honey and an admixture of PA honey and NPA honey.

52) The method of claim 42, and further comprising adding a chemical preservative to the therapeutic material formed in step d).

53) The method of claim 42, and further comprising the step of adding to the therapeutic material of step d) a honey selected from the group consisting of PA honey, NPA honey, and an admixture of PA honey and NPA honey.

54) The method of claim 44, and further, after separation step e), mixing some of the separated fluid back in with the micronized AM lysates so that the resultant combination of therapeutic material is of sufficiently low viscosity as to permit it to be used as a therapeutic fluid to be applied to diseased tissue.

55) The method of claim 44, and further comprising the step of combining micronized AM lysates that have been separated from the micronizing mixture with a physiologic solution to the consistency of a paste for manipulation into a 3D device for therapeutic application diseased tissue.

56) The method of claim 55, and further comprising pre-selecting a specific particle size of the micronized AM lysates separated from the micronizing mixture before combining the pre-selected AM lysates with the physiologic solution to the consistency of a paste.

57) The method of claim 43, and further comprising the step of dehydrating the mixture of physiologic solution and micronized AM tissue of step d) to the consistency of a paste for manipulation into a 3D device for therapeutic application to diseased tissue.

58) The method of claim 43, and further comprising the step of dehydrating the mixture of physiologic solution and micronized AM tissue of step d) completely, to the consistency of granules for storage and shipment to the practitioner.

59) The method of claim 55, wherein the physiologic solution is an aqueous mixture of honey selected from the group consisting of PA honey, NPA honey, and an admixture of PA honey and NPA honey.

60) The method of claim 55, and further comprising the step of forming the mixture into a 3D device of a pre-selected size and shape suitable for application to a particular diseased tissue.

61) The method of claim 59, and further comprising the step of forming the mixture of step d) into a 3D device by molding the mixture.

62) The method of claim 60, and further comprising the step of fenestrating the 3D device by using a mold with protruberances to puncture the 3D device material being molded and thereby facilitate dehydration thereof.

63) The method of claim 59, and further comprising the step of dehydrating the formed 3D device prior to storage and shipping.

64) The method of claim 62, and further comprising the step of rehydrating the formed 3D device before application thereof to diseased tissue.

65) The method of claim 62, and further comprising the step of rehydrating the formed 3D device with a solution including a substance selected from the group consisting of honey, honey derivatives, pharmaceuticals, pharmaceutical antibiotics and anti-inflammatory drugs, hyaluronic acid, cross-linked hyaluronic acid, chondroitin sulfate, cross-linked chondroitin sulfate, and anti-inflammatory fluids filtered from micronized amniotic membrane lysates.

66) The method of claim 62, and further comprising the step of rehydrating the formed 3D device with a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

67) The method of claim 59, and further comprising the step of shaping the therapeutic material into an ocular contact lens.

68) The method of claim 59, and further comprising the step of pressing the micronized AM lysates into a sheet form.

69) The method of claim 67, and further providing the step of puncturing the sheet of filtered AM lysates during the pressing step of production so that the 3D therapeutic device is fenestrated to facilitate dehydration.

70) The method of claim 68, and further comprising the steps of dehydrating the fenestrated sheet therapeutic material, and then coating the dehydrated sheet of therapeutic material with honey.

71) The method of claim 67, and further comprising the step of lyophilizing the honey into an anhydrous powder form, and then coating the dehydrated sheet of therapeutic material with the powdered honey.

72) The method of claim 67, and wherein the honey used for coating the sheet of therapeutic material is an aqueous mixture of honey; and further comprising the step of dehydrating the combined honey-coated sheet of therapeutic material.

73) The method of claim 55, and further comprising the steps of freezing and storing the therapeutic material in a frozen state.

74) The method of claim 55, and further comprising the steps of adding a chemical preservative to the physiologic solution and AM lysate paste.

75) The method of claim 67, and further comprising repeating the step of pressing the micronized AM lysate into a sheet form, thereby forming multiple sheets of the 3D therapeutic material, and then stacking multiple sheets of the therapeutic material to form a multi-ply block of a preselected thickness, and shaping the multi-ply block by a method selected from the group consisting of rolling, cutting, carving, molding, pressing, and 3D printing into a preselected 3D form for therapeutic application to diseased tissue.

76) The method of claim 74, and further comprising the step of dehydrating the multi-ply block of therapeutic material for shipping and storage.

77) The method of claim 75, and further comprising the step of rehydrating the 3D device before use.

78) The method of claim 75, and further comprising the step of rehydrating the 3D device before use with a solution including at least one chemical selected from the group consisting of honey, honey derivatives, pharmaceuticals, pharmaceutical antibiotics and anti-inflammatory drugs, hyaluronic acid, cross-linked hyaluronic acid, chondroitin sulfate, cross-linked chondroitin sulfate, and anti-inflammatory fluids filtered from micronized amniotic membrane lysates.

79) The method of claim 75, and further comprising the step of rehydrating the 3D device before use with a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

80) A three-dimensional (3D) device for treatment of diseased tissues by application of the 3D device to such diseased tissues, the 3D device comprising at least two sheets of dehydrated intact AM tissue, wherein the stack of at least two sheets of intact dehydrated intact AM tissue is of sufficient thickness to be further formed to a preselected 3D shape for a specific therapeutic application.

81) The 3D device of claim 80, wherein the at least two sheets are dehydrated, coated with honey and dehydrated again so that each sheet of dehydrated intact AM tissue is coated with anhydrous honey powder, wherein the honey is selected from the group consisting of Non-Peroxide Activity (NPA) honey, Peroxide Activity (PA) honey, and an admixture of PA honey and NPA honey.

82) The 3D device of claim 80, and further comprising an adhesive substance between any two adjacent sheets of the at least two substantially planar sheets of dehydrated intact AM tissue.

83) The 3D device of claim 82, wherein the adhesive substance between any adjacent sheets of the at least two sheets of dehydrated intact AM tissue is comprised of honey.

84) The 3D device of claim 80, and further wherein the 3D device is rehydratable.

85) The 3D device of claim 80, and further wherein the 3D device is rehydratable with a solution including at least one substance selected from the group consisting of honey, honey derivatives, pharmaceuticals, pharmaceutical antibiotics and anti-inflammatory drugs, hyaluronic acid, cross-linked hyaluronic acid, chondroitin sulfate, cross-linked chondroitin sulfate, and anti-inflammatory fluids filtered from micronized amniotic membrane lysates.

86) The 3D device of claim 80, and further wherein the 3D device is rehydratable with a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells.

87) The 3D device of claim 80, wherein at least one sheet of the at least two sheets of dehydrated intact AM tissue is marked to indicate intended direction of orientation of the 3D device during application to diseased tissue.

88) The 3D device of claim 82, wherein the outermost sheets of the at least two sheets of dehydrated intact AM tissue are positioned with the basement membrane of said outmost sheets facing away from each other, such that the 3D device can be rolled into the form of a tube, the tube having basement membrane on the exterior wall and on the lumen thereof, to thereby provide a device for use in repair or replacement of blood vessels or other tubular diseased tissue.

89) The 3D device of claim 88, wherein the 3D device is dehydratable and rehydratable with a solution including at least one substance selected from the group consisting of honey, honey derivatives, pharmaceuticals, pharmaceutical antibiotics and anti-inflammatory drugs, hyaluronic acid, cross-linked hyaluronic acid, chondroitin sulfate, cross-linked chondroitin sulfate, and anti-inflammatory fluids filtered from micronized amniotic membrane lysates.

90) The 3D device of claim 88, and further comprising a chemical preservative.

91) The 3D device of claim 88, wherein the 3D device is dehydratable and rehydratable with a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, plasma cells, and vascular endothelial cells.

92) A three-dimensional (3D) device for treatment of diseased tissues by application of the 3D device to such diseased tissues, the device being comprised of micronized amniotic membrane (AM) tissue in a physiologic solution and dehydrated to a paste-like consistency for manipulation into a specific preselected form, wherein the physiologic solution with the micronized amniotic membrane tissue comprises an aqueous mixture of honey and a chemical preservative, and further wherein the dehydrated paste is molded into the form of an ocular contact lens for deployment on the surface of a diseased eye, the ocular contact lens having a convex surface coated with an antibiotic and a concave surface coated with honey.

93) A three-dimensional (3D) device for treatment of diseased tissues by application of the 3D device to such diseased tissues, the device being comprised of micronized amniotic membrane (AM) tissue in a physiologic solution and dehydrated to a paste-like consistency for manipulation into a specific preselected form, wherein the physiologic solution with the micronized amniotic membrane tissue comprises an aqueous mixture of honey selected from the group consisting of Non-Peroxide Activity (NPA) honey, Peroxide Activity (PA) honey and an admixture of PA honey and NPA honey, wherein the aqueous mixture of honey further comprises a chemical preservative, wherein the 3D device is pressed into at least one sheet having a selectively variable thickness, and further wherein the at least one sheet is a stack of at least two sheets of sufficient thickness to be further formed to a preselected 3D shape for a specific therapeutic application, and further comprising an adhesive substance between adjacent sheets of the at least two sheets of micronized AM tissue, wherein the adhesive substance between any two adjacent sheets of the at least two sheets of micronized AM tissue is comprised of honey, and further wherein the sheets of the at least two sheets are dehydrated, coated with honey and dehydrated again, and then stacked, for shipping and storage so that the at least two sheets of micronized AM tissue are coated with anhydrous honey powder selected from the group consisting of NPA honey, PA honey and an admixture of PA honey and NPA honey, and further wherein the 3D device is rehydratable with a solution including at least one substance selected from the group consisting of honey, honey derivatives, pharmaceuticals, pharmaceutical antibiotics and anti-inflammatory drugs, hyaluronic acid, cross-linked hyaluronic acid, chondroitin sulfate, cross-linked chondroitin sulfate, and anti-inflammatory fluids filtered from micronized amniotic membrane lysates, and further wherein the 3D device is rehydratable with a solution including at least one substance including cells selected from the group consisting of circulating peripheral blood cells, bone marrow cells, stem cells, structural cells (bone, skin, hair, cartilage, tooth), and immune effector cells, including T-cells, B-cells, natural killer cells, macrophages, and plasma cells, and further wherein the 3D device is marked on an outer surface thereof to indicate direction of positioning on the diseased tissue.
